(19) United States
(12) Patent Application Publication
(10) Pub. No.: US 2007/0043597 A1
(43) Pub. Date: Feb. 22, 2007

(54) Physiology network and workstation for use therewith

(75) Inventor: Brenda Donaldson, Harrison Township, MI (US)

Correspondence Address: THE SMALL PATENT LAW GROUP LLP, 611 OLIVE STREET, SUITE 1611, ST. LOUIS, MO, 63101, US

(73) Assignee: GENERAL ELECTRIC COMPANY

(21) Appl. No.: 11/433,952

(22) Filed: May 15, 2006

Related U.S. Application Data
(63) Continuation-in-part of application No. 11/204,590, filed on Aug. 16, 2005.

(51) Int. Cl. G06F 19/00; A61B 5/00; A61B 8/00

(52) U.S. Cl. 705/3; 600/437

(57) ABSTRACT

A method for managing and distributing information over a network joined to a database includes using a local workstation and an associated ultrasound system in a procedure room during a physiology procedure to obtain physiology signals from the subject, including ultrasound signals. A remote workstation in a control room is used to operate the ultrasound system remotely, so that a person in the control room can control the ultrasound system while receiving, processing, and displaying the ultrasound signals obtained from the subject in real time, while an ultrasound procedure is being performed on the subject. The physiology signals are processed at the remote workstation in real-time during the physiology study. A pre-recorded patient record associated with the subject is requested from the database. The database is accessed to obtain the pre-recorded patient record associated with the subject. The physiology workstation is provided the patient record associated with the subject.

### CROSS-REFERENCE TO RELATED APPLICATIONS

This application is a continuation-in-part of U.S. patent application Ser. No. 11/204,590, filed Aug. 15, 2005, entitled “Physiology network and workstation for use therewith”, which is hereby incorporated by reference in its entirety.

### BACKGROUND OF THE INVENTION

Embodiments of the present invention generally relate to a physiology network and a workstation configured to operate with a hospital/medical network. More particularly, embodiments relate to a physiology workstation that operates to co-display images and physiology information acquired during a physiology procedure as well as pre-recorded patient information obtained from a patient information database.

Various types of physiology workstations have been proposed such as electrophysiology (EP) workstations, hemo-dynamic (HD) workstations, and the like. Heretofore, physiology workstations operate independent and distinct from other equipment and systems utilized during the physiology study, such as a fluoroscopy system, an ultrasound system, an ablation system, a cardiac mapping system and the like. Generally, EP, HD and ablation procedures are carried out in a procedure room including, among other things, EP catheters, HD catheters and patient sensors joined to an EP or HD workstation. The procedure room also includes a fluoroscopy system, a diagnostic ultrasound system, a stimulator, a patient monitoring device and an ablation system. A monitoring room and a control room may be located adjacent to the procedure room. The procedure room may include a magnetic catheter guidance system such as those available from Stereotaxis, Inc., St. Louis, Mo.

Also, conventional physiology workstations operate independent and distinct from other equipment and systems distributed through a medical or hospital network. Conventional physiology workstations do not present, and do not afford access to, historic patient information, such as patient records. Instead, while a hospital/medical system may locally store different types of patient records, today such patient records are not accessible at a physiology workstation.

Numerous hospital/medical systems exist around the United States and around the world. These hospital/medical systems range in the degree that equipment and systems store patient records and are connected to one another. For example, local databases may exist within different functional areas of a hospital/medical network, such as the emergency room, patient recovery rooms, laboratories, diagnostic imaging facilities, operating rooms and the like. The functional areas collect certain overlapping patient information and certain unique patient information. Examples of patient information include patient demographic information, interventional medical procedure history, physician/lab reports, past measured physiologic performance, diagnostic images and reports, prior physiology studies and the like.

However, conventional physiology workstations operate independent and distinct from other equipment and systems distributed throughout the medical or hospital network. Conventional physiology workstations do not present, and do not afford access to, historic patient information, such as patient records or previously acquired computerized axial tomographies, magnetic resonance images, or other diagnostic images. Instead, while a hospital/medical system may locally store different types of patient records, today such patient records are not accessible at a physiology workstation.

Some known physiology workstations have the ability to view images obtained from other systems such as an ultrasound system. However, these physiology workstations remain separate from the ultrasound systems from which they obtain images in that neither the ultrasound system nor the physiological workstation are capable of controlling any functions of the other system.

### BRIEF DESCRIPTION OF THE INVENTION

Therefore, in one aspect, some configurations of the present invention provide a physiology network configured to operate with a medical network. The physiology network includes an ultrasound system that has an ultrasound probe. Also provided is a local workstation configured to operate in a procedure room with the ultrasound system and operatively coupled to the ultrasound system to display ultrasound signals obtained from a subject during an ultrasound procedure carried out on the subject. The local workstation has a network interface configured to communicatively couple to the medical network. Also provided is a database storing patient records associated with the subject undergoing the physiology procedure, a server, operatively coupled to the medical network and the database, for managing and controlling access to the database. The server is configured to provide, to the local workstation and to a remote workstation, a patient record associated with the subject. The local workstation co-displays the ultrasound signals and information from the patient record to an operator of the local workstation. Also provided is a remote workstation configured to operate in a control room different from the procedure room and configured to operate the ultrasound system remotely, so that a person in the control room can control the ultrasound system while receiving, processing, and displaying the ultrasound signals obtained from the subject in real time, while an ultrasound procedure is being performed on the subject. The remote workstation includes an EP workstation, an HD workstation, or a combination EP/HD workstation.

In another aspect, some configurations of the present invention provide a method for managing and distributing patient and physiology information over a network joined to a database. The method includes using a local workstation and an associated ultrasound system in a procedure room during a physiology procedure to obtain physiology signals from the subject, wherein the physiology signals include ultrasound signals. The method further includes using a remote workstation in a control room different from the procedure room to operate the ultrasound system remotely, so that a person in the control room can control the ultrasound system while receiving, processing, and displaying the ultrasound signals obtained from the subject in real time, while an ultrasound procedure is being performed on the subject. Also included is processing the physiology signals at the remote workstation in real-time during the physiology study, requesting from the database a pre-recorded patient record associated with the subject, the pre-recorded patient record being generated and stored prior to the physiology procedure, accessing the database to obtain the pre-recorded patient record associated with the subject, and providing, to the physiology workstation, the patient record associated with the subject.

Advantageously, configurations of the present invention co-display, at a remote workstation, the same information as at a local workstation and permit an operator of the remote workstation to update patient information, patient logs, and the like during the procedure.

### BRIEF DESCRIPTION OF THE DRAWINGS

FIG. 1 illustrates a block diagram of a hospital/medical network joined with a physiology workstation in accordance with an embodiment of the present invention.

FIG. 2 illustrates a graphical representation of a database architecture for storing patient records and files in accordance with an embodiment of the present invention.

FIG. 3 illustrates a flowchart of the process carried out to utilize prerecorded patient information in combination with real-time physiology data obtained during a physiology procedure.

FIG. 4 illustrates a block diagram of the functional modules operating in order to utilize prerecorded patient information in combination with real-time physiologic procedure information.

FIG. 5 illustrates an exemplary data packet processing sequence for packet sizing and conveying patient files over the network in accordance with an embodiment of the present invention.

FIG. 6 illustrates a block diagram of a networked physiology workstation formed in accordance with an embodiment of the present invention.

FIG. 7 illustrates a block diagram of ablation and imaging equipment joined to the networked physiology workstation in accordance with an embodiment of the present invention.

FIG. 8 illustrates an exemplary window layout for a configuration of monitors for a networked physiology workstation formed in accordance with an embodiment of the present invention.

FIG. 9 illustrates a block diagram of a networked image management system formed in accordance with an embodiment of the present invention.

FIG. 10 illustrates a block diagram of a physiology network having a remote physical keyboard and formed in accordance with an embodiment of the present invention, wherein the remote physical keyboard provides keys corresponding to all or nearly all the functionality of an associated ultrasound system.

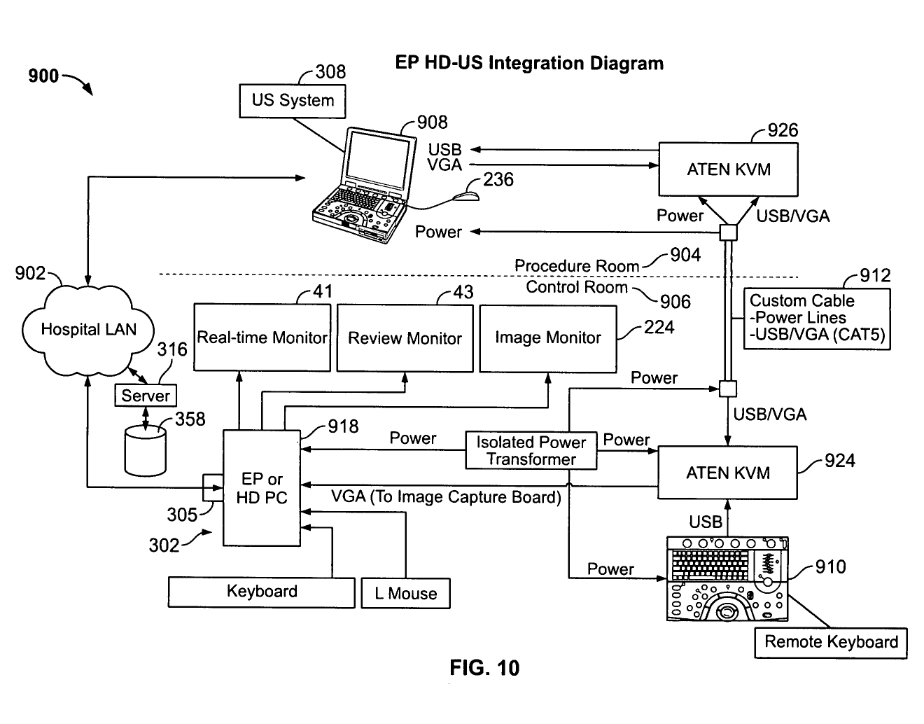

FIG. 11 illustrates a block diagram of a physiology network having a remote keyboard configured to communicate with the ultrasound system via a wired or wireless connection separate from a medical network.

FIG. 12 illustrates a block diagram of a physiology network having a visual keyboard simulator software module configured to run, at least in part, on the remote (physiological) workstation, to display a simulated keyboard on an image monitor, and to communicate with the local (ultrasound) workstation to thereby control the ultrasound system. Also, the remote physical keyboard is a standard PC-style keyboard having either fewer or different keys than the remote physical keyboard shown in FIG. 10.

FIG. 13 illustrates a block diagram of a physiology network having a visual keyboard simulator software module configured to run, at least in part, on the remote (physiological) workstation, to display a simulated keyboard on a review monitor, and to communicate with the local (ultrasound) workstation to thereby control the ultrasound system.

FIG. 14 illustrates a block diagram of a physiology network having a visual keyboard simulator software module configured to run, at least in part, on the remote (physiological) workstation, to display a simulated keyboard on an image monitor, and to communicate with the local (ultrasound) workstation to thereby control the ultrasound system. A remote keyboard that is configured to communicate signals other than control signals to the ultrasound system via a wired or wireless connection separate from a medical network is also provided.

FIG. 15 illustrates a block diagram of a physiology network wherein the remote keyboard is directly connected to the ultrasound system via a wired or wireless connection other than the medical network.

### DETAILED DESCRIPTION OF THE INVENTION

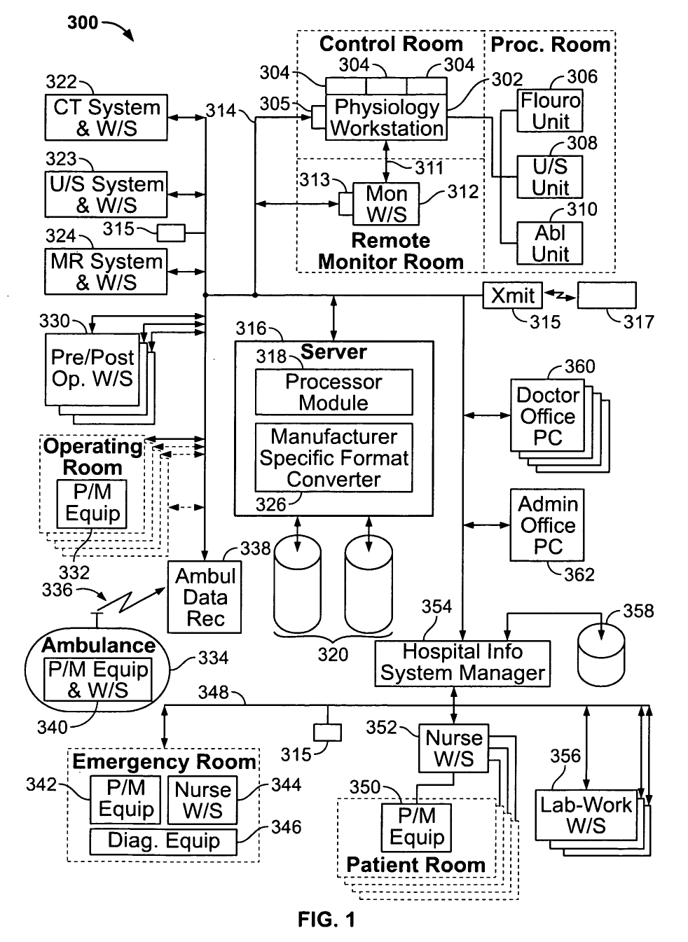

FIG. 1 illustrates a hospital/medical system having a network 300 joined with a physiology workstation 302 in accordance with an embodiment of the present invention. The network 300 may represent one or more, or a combination of, a local area network, a wide area network a Token Ring network, and ethernet network, a fiber distributed data interface and the like. The network 300 may also support text message capabilities and a Voice Over IP Protocol. The physiology workstation 302 includes multiple monitors 304 for presenting various types of patient and physiology information. The physiology workstation 302 may be located in the procedure room or in a separate control room and communicatively joined to various systems in the procedure room, such as a fluoroscopy system 306, an ultrasound system 308, and an ablation system 310, all of which operate as explained above. The physiology workstation 302 processes and displays the physiology signals obtained from a subject during a physiology procedure carried out on the subject in the procedure room. The physiology workstation 302 is joined to a network interface 305 that is connected to a network link 314. The network interface 305 is assigned a unique Internet protocol (IP) address that is uniquely associated with the physiology workstation 302. In one embodiment, the IP addresses are static, namely the IP addresses are assigned to various devices at the time that the device is added to the network. Alternatively, the IP addresses may be assigned to various devices dynamically by the server and changed periodically.

The physiology workstation 302 is joined over the network link 314 to a server 316 that coordinates and manages data transfer and data communication over at least a portion the network 300. The server 316 includes a processor module 318 that stores and retrieves patient records to and from a database 320. The database with the subject undergoing the physiology procedure. The server 316 manages and controls access to the database 320 to, among other things, provide to e 320 stores patient records that may include one or more records associated the physiology workstation, patient records and/or files associated with the subject. The physiology workstation co-displays the physiology signals and the patient information from the patient records/files for viewing and analysis by an operator at monitors 304.

The medical/hospital system includes numerous functional areas, such as an emergency room, patient recover rooms, laboratories, physician's offices, operating rooms, diagnostic examination rooms, administrative offices and the like. The emergency room includes, for example, patient monitoring equipment 342, a monitoring/control workstation 344 and diagnostic equipment 346. The patient monitoring equipment 342 and diagnostic equipment 346 obtain patient information, while the workstation 344 coordinates and controls transfer of patient information to/from the monitoring equipment 342 and diagnostic equipment 346. The workstation 344 also allows an operator to enter other patient information, including basic demographic information. Optionally, the workstation 344 may transfer the patient information over link 348 to a hospital information system manager 354 which directs the patient information to database 356 and/or server 316. Alternatively, the workstation 344 may be directly joined to the network 300 and have a unique internet protocol (IP) address within the network 300 in order to transfer directly patient information onto the network 300 from the diagnostic equipment 346 and patient monitoring equipment 342.

The patient rooms also include patient monitoring equipment 350 joined with workstations 352 that are in turn joined to the hospital information system manager 354 over link 348, and/or directly to the network 300. Workstations 356 are also provided in the labs to facilitate entry of patient information associated with lab reports. The lab reports are conveyed over link 348 to the hospital information system manager 354 and/or directly over the network 300 to the server 316. When directly joined to the network 300, the workstations 352 and 356 are statically or dynamically assigned unique internet protocol (IP) address within the network 300 and control direct transfer of patient information onto the network 300. Optionally, the hospital information system manager 354 may store the patient information from the emergency room, patient rooms and the labs in the local database 358. In addition or alternatively, the hospital information system manager 354 may communicate with the server 316 to store the patient information in database 320.

The physician's offices are also provided with computers 360 and the hospital administrator's offices are provided with computers 362. Computers 360 and 362 are joined to the network 300 to retrieve, modify and enter patient information through the server 316 and database 320. The computers 360 and 362 permit real-time monitoring of, and consultation in connection with, procedures being conducted throughout the network 300, including the physiology procedure. The consultation may be provided through textual and/or audio messages exchanged between the physiology workstation or remote monitoring workstation 312, and one of computers 360 and 362. The text consultation may be provided through a “same time” text messaging format. The audio consultation may be provided through a Voice Over IP Protocol supported by the network 300, hospital information system manager 354 and server 316.

Optionally, the network 300 may include local wireless transmitters 315 distributed throughout the medical/hospital system. The transmitters 315 support bidirectional local transmission, throughout the medical/hospital facility, of physiology signals, diagnostic images and other patient information. Physicians and other personnel may be provided with wireless portable hand-held devices 317 having text and graphic display and entry capabilities (such as personal digital assistants, cell phones, laptop computers and the like). The hand-held devices 317 enable the physicians and other personnel to monitor patients (e.g., during a physiology procedure) while roaming about the medical/hospital facility. The wireless hand-held devices 317 may include a transmitter and microphone and/or keypad supporting audio and/or text entry to enable the physician or other personnel to provide feedback, consultation and the like, such as to the operator of the physiology workstation and the team conducting a physiology procedure.

The patient records are not limited to the specific types of data discussed herein, but instead may vary. By way of example only, the patient records may include at least one of patient demographic information, interventional medical procedure history, physician/lab reports, past measured physiologic performance, and diagnostic image information, and prior physiology studies. The interventional medical procedure history may include, among other things, an interventional medical history of the patient representing a radiology report, cardiology report, implanted device report and the like. The implanted device report identifies, among other things, implanted device parameters and settings. The physician/lab reports may include, among other things, a physician office report, a lab-work report, medication subscribed to the subject and the like. The patient record may include pre-recorded stored ECG traces recorded prior to the physiology procedure.

The physiology workstation 302, monitoring workstations 312,344,352,356, computers 360,362, and hand-held devices 317 may co-display the pre-recorded stored ECG traces and real-time ECG traces, wherein the real-time ECG traces are obtained from the physiology signals obtained from the subject during the physiology procedure. Also, the patient record may include a pre-recorded prior physiology study and/or case log. The physiology workstation 302, monitoring workstations 312,344,352,356, computers 360,362, and hand-held devices 317 may co-display the pre-recorded prior physiology study and a real-time physiology study obtained from the subject during the physiology procedure. Alternatively, physiology workstation 302, monitoring workstations 312,344,352,356, computers 360,362, and hand-held devices 317 may provide co-display by presenting, on one monitor, prerecorded patient information, and on another monitor, real-time information (e.g. ECG and EP signals, live diagnostic images, earlier diagnostic images recorded during the procedure).

The patient record may be generated and periodically updated throughout the life of the patient as the patient undergoes various examinations, procedures, studies and the like. For example, the patient record may be updated with patient monitoring information such as obtained while in an ambulance or while obtained in the emergency room of a hospital. The patient record may include prerecorded diagnostic images, such as obtained from a CT system 322, an ultrasound system 323 and an MR system 324 located within the hospital network 300. Other examples of diagnostic images may be obtained from PET and SPECT systems. The CT, ultrasound and MR systems 322-324 also include network interfaces having IP addresses for each system to facilitate transfer of images and other data over the network 300. Further, the patient records may include patient monitoring information that is recorded prior to the procedure (e.g. prerecorded), such as by patient monitoring equipment. The patient monitoring equipment may be located anywhere throughout the medical network, such as in an ambulance, and emergency room, a patient recovery room, in operating room, a physician's office and the like.

The server 316 also includes a manufacturer specific format converter 326 that facilitates conversion of images and other patient information between formats specific to different manufacturers of diagnostic imaging equipment. For example, CT system 322 may be manufactured by one company, while ultrasound system 323 and the physiology workstation 302 are manufactured by a different second manufacturer. In certain instances, the images generated by the CT system 322 are formatted in a manner different from the formats supported by the ultrasound system 323 and physiology workstation 302. In this instance, the processor 318 may be configured to identify potential formatting compatibility problems. When a formatting incompatibility arises, the converter 326 may be utilized to transform the data (e.g. image files and the like) from one manufacturer specific format to a format known to be compatible with the physiology workstation 302.

The physiology workstation 302 generates a physiology study file(s) (including case log, physiology signals, EP mapping information and the like) throughout the procedure and, upon the completion of the procedure, exports the physiology study file(s) over the hospital network 300. The completed physiology study file(s) may be stored in the database 320 by the server 316 and/or remotely conveyed to a third-party application, such as to build graphic reports from the physiology study. The completed physiology study file(s) may be later viewed at the physiology workstation 302, monitoring workstations 312,344,352,356, computers 360,362, and hand-held devices 317.

A separate monitoring room may be provided, in which a remote monitoring workstation 312 is located. The remote monitoring workstation 312 permits the operator to view all or at least a portion of the information displayed at the monitors 304 and at each of systems 306, 308 and 310. The remote monitoring workstation 312 may co-display information from a patient record and physiology signals obtained from a subject during a physiology procedure, such that the remote monitor 312 presents the same information as displayed on the monitors 304 of the physiology workstation 302. The monitoring workstation 312 also supports data entry by the operator, such as to permit a case log associated with the particular physiology procedure to be updated during the procedure by the operator. The monitoring workstation 312 may communicate directly with the physiology workstation 302 over a link 311. In addition or alternatively, the monitoring workstation 312 may include a network interface 313 (such as used to define a static or dynamic IP address for the workstation) through which images, records, data and the like are conveyed over the network 300 and/or to/from the physiology workstation 302.

As shown in FIG. 1, various workstations, computers and other systems may be joined to the network 300. For example, workstations 330 may be provided for hospital personnel to perform pre-operative and post operative planning, reporting, diagnosis and the like. In addition, patient monitoring equipment 332, located in operating rooms, may be joined to the network 300, in order to provide patient monitoring information to update the patient records. An ambulance 334 is generally illustrated to have a wireless link 336 to the data receiver 338 that is joined to the network 300. The ambulance 334 includes patient monitoring equipment 340 that monitors and records patient information while in transit to a hospital. Upon arrival at the hospital, as the patient is being conveyed into the hospital, the patient monitoring equipment 340 may convey wirelessly the patient information over the link 336 to the ambulance data receiver 338. The ambulance data receiver 338 conveys the patient information over the network 300 to the server 316.

Optionally, to reduce the bandwidth needs of the network, the monitoring workstations 312,344,352,356, computers 360,362, and hand-held devices 317 may be configured to receive and display a streaming video of all or a portion of the information or windows displayed on the monitors 304 of the physiology workstation 302. For example, the monitoring workstation 312 may include three monitors and the operator may choose to display the complete content of the three monitors 304 provided at the physiology workstation. For example, the operator of monitoring workstation 312 may choose to order the windows in a different layout than the window layout on monitors 304. For example, the operator of workstations 344,352,356 may designate particular windows of interest, such as only the real-time physiology signals, and/or the real-time fluoroscopy or ultrasound images. For example, the operators of computers 360,362 and hand-held devices 317 may choose to only view a single window. Optionally, the operators of any workstation, computer or hand-held devices 317 may choose only to be notified when certain parameters of the patient undergoing the procedure exceed or fall below certain predetermined thresholds (set by the procedure team or the operator of the particular workstation, computer or hand-held device).

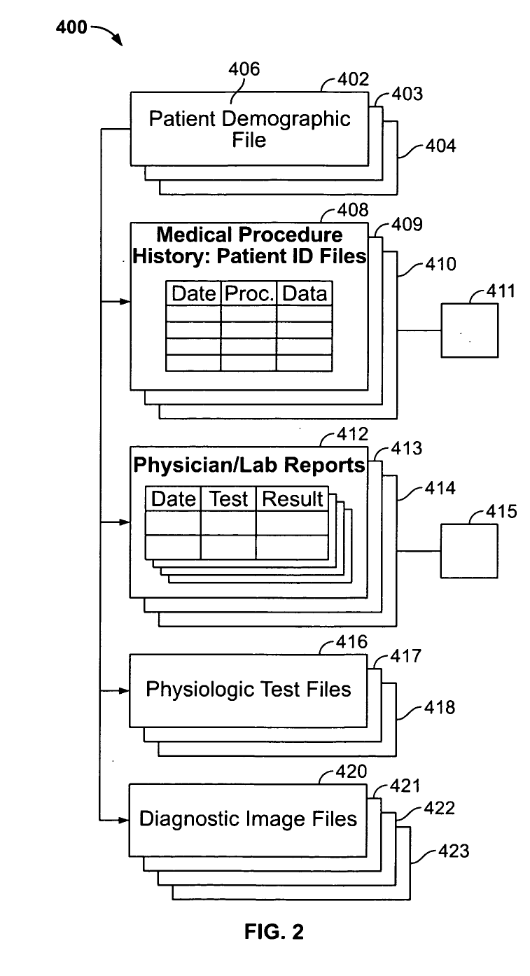

FIG. 2 illustrates a pictorial representation of a relational database 400 comprised of records and files. The term “record” refers to one or more electronic documents having a relation with one another, such as relating to a single individual or patient. The term “file” refers to an individual electronic document. The patient records 400 stored in database 320 include numerous records associated with the patient base of the hospital network, hospital system and the like. The database 320 and network may be stored in one location or divided between numerous locations and distributed throughout the United States and around the world. The database 320 may include patient records from independent hospitals, HMOs, PPOs, and other medical organizations, as well as from universities, research institutes and the like.

FIG. 2 illustrates a plurality of patient records 402-404. Patient record 402 includes a patient demographic file 406 which is stored in a one-to-one relation with multiple medical procedure history files 408-410, physician/lab report files 412-414, physiologic test files 416-418, and diagnostic image files. All of files 406-418 are associated with the same patient. The patient demographic file 406 and files 408-418 may be identified by a single patient ID, such as patient name, Social Security number, medical insurance ID and the like. The patient demographic file 406 may record basic information, such as name, weight, height, age, race, parental history and the like. The medical procedure history file 408 may record information associated with the particular procedure, such as the date of the procedure, type of procedure and other procedure specific information. The medical procedure history files 408-410 may also be linked to video files 411 containing graphic and video information. For example, when medical procedure history file 410 corresponds to a colonoscopy, the video file 411 may include a video recording of the colonoscopy. Alternatively, when the medical procedure history file corresponds to surgery, such as bypass surgery, heart valve surgery and the like, the video file 411 may include a recording of the internal cardiac images captured before, during and/or after the surgery.

The physician/lab reports 412-414 include information such as the date of a physician examination, type of examination and results, or the date of lab work, type of lab work and conclusions. In the event that blood samples and other biologic samples are analyzed, the physician/lab reports may also be joined with video or image files 415 the examined tissue, blood sample and the like. The video or image may be captured by sophisticated diagnostic equipment. The physiologic test files 416-418 may correspond to stress tests and the like. The diagnostic image files 420-423 may correspond to CT, ultrasound, MR, PET, SPECT images (2D, 3D, 4D) and the like.

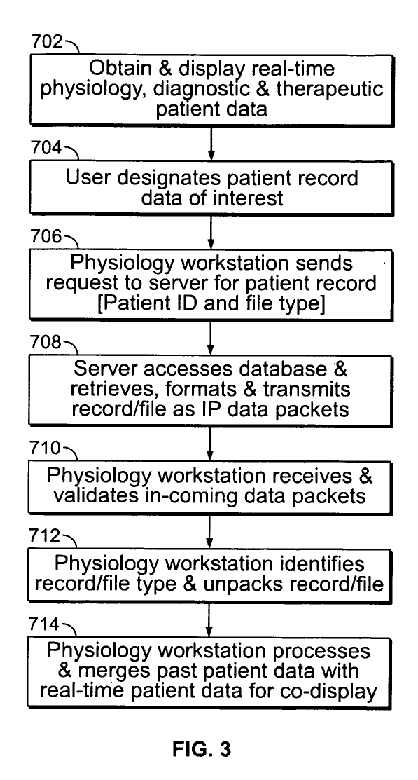

FIG. 3 illustrates a flowchart of the process carried out to utilize prerecorded patient information in combination with real-time physiology data obtained during a physiology procedure. At 702, the physiology workstation 302 obtains and displays real-time physiology signals, diagnostic images, therapeutic patient data and the like. At 704, the operator of the physiology workstation 302 accesses the network 300 and designates a patient record of interest. At 706, the physiology workstation 302 conveys the request to the server 316 for the complete patient record or select patient files.

The patient may be identified may be based on the patient ID, as well as an identification of one or more patient files of interest. For example, the operator may enter the patient's social security number and request the patient demographic information. The physiology workstation would then automatically populate the fields of the physiology study contained within the patient demographic information. For example, the physiology study may include a patient name field, patient age field, insurance carrier information field, billing address field and the like. To the extent that the above fields are completed within the patient's demographic information, the physiology workstation automatically populate such fields in the current physiology study, thereby reducing the study data entry time of the operator. As another example, the operator may request any prior physiology studies conducted upon the present patient, as well as any pre-existing diagnostic images of the patient's cardiac system. The physiology workstation may then present the prior physiology study on one monitor next to a second monitor displaying the real-time physiology study.

At 708, the server 316 receives the request and accesses the database 320 to retrieve the requested record or files. The server 316 performs the request based upon the patient ID and record/filed designators. The server 316 also determines whether formatting incompatibilities exist between the stored patient record format and the formats supported by the physiology workstation 302. When formatting incompatibilities exist, the data from the database 320 is passed through the converter 326 to be reformatted prior to being transmitted to the physiology workstation 302. By way of example, the patient record may be formatted in data packets associated with an Internet protocol (for example TCP/IP). The server 316 records within the stream of data packets the IP address of the intended receiver, as well as the patient identifier and record/file types.

At 710, the network interface 305 receives data packets and determines that the data packets are intended for the physiology workstation 302 (based on the IP address header information within the data packets). At 712, the workstation 302 validates and unpacks the incoming patient record/file (such as by comparing the patient ID and filed designators to the patient ID of the subject of the physiology study and of the requested files). At 714 the physiology workstation 302 processes and merges the past patient information with the real-time physiology signals for co-display.

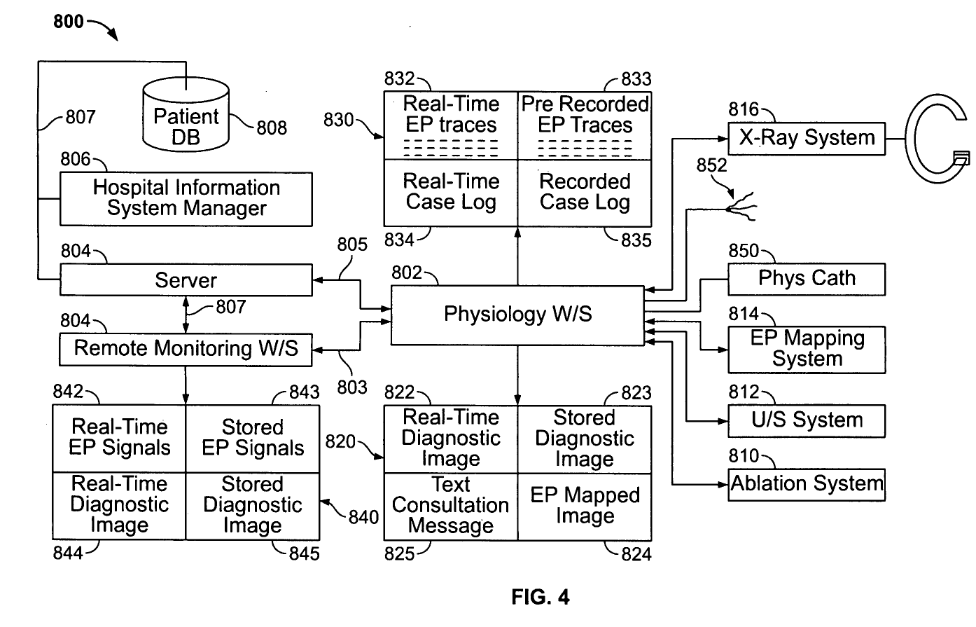

FIG. 4 illustrates a block diagram of a physiology network 800 that includes a physiology workstation 802 utilized to carry out physiology studies (e.g., electrophysiology procedures, hemo-dynamic procedures and the like). The physiology workstation 802 is connected to an ablation system 810, an ultrasound system 812, an EP mapping system 814 and an x-ray system 816. The physiology workstation 802 is joined through a network 805 to a server 804 which in turn communicates over a network 807 with a hospital information system manager 806 and a patient information database 808. The physiology workstation 802 communicates with the server 804 to obtain patient records and/or patient files, from which patient information is extracted, formatted and presented on one or more of the monitors 820, 830 and 840. In some configurations of the present invention, demographics are or can be inserted or imported into physiology workstation 802 and then pushed to or pulled into ultrasound system 812.

The physiology workstation 802 displays, on monitor 820, various windows such as real-time diagnostic images 822 from the ultrasound system 812 (surface, IVUS and the like) and x-ray system 816 during the procedure. The monitor 820 may also include a window that displays prerecorded diagnostic images 823 which are obtained prior to the physiology procedure. The monitor 820 also may include a window that displays EP mapped images 824 which represent virtual representations developed based upon the data points taken by the EP mapping system 814. The monitor 820 also includes a window that displays text consultation messages 825 that may be conveyed to the physiology workstation 802 over the network link 805. The text consultation messages 825 may be sent during the physiology procedure from a physician located remote from the procedure room, such as from a personal computer of a physician specialist and the like.

The physiology workstation 802 displays in discrete windows, on monitor 830, real-time physiology traces 832 (EP or HD), a real-time case log 834, prerecorded physiology traces 833 and a prerecorded case log 835. The real-time physiology traces 832 and real-time case log 834 are generated by the physiology workstation 802 during the physiology procedure based on signals from physiology catheters 850 (EP or HD) and ECG electrodes 852. The prerecorded physiology traces and case log 833 and 835 are generated during a prior physiology procedure by the physiology workstation 802 or by a different physiology workstation.

In the exemplary embodiment of FIG. 4, the real-time and prerecorded physiology traces and case logs are co-displayed on a common monitor 830. Alternatively, the real-time and prerecorded physiology traces and case logs may be co-displayed on separate, but closely positioned monitors. Similarly, the real-time diagnostic images and stored diagnostic images 822 and 823, text consultation message 825 and EP mapped images 824 may be co-displayed on a common monitor are co-displayed on separate but closely positioned monitors.

The physiology workstation 802 communicates over link 803 with a remote monitoring workstation 804. The monitoring workstation 804 includes one or more monitors 840 configured to display all or at least a portion of the windows displayed on monitors 820 and 830. In the example of FIG. 4, The monitor 840 displays in various windows real-time physiology signals 842, stored physiology signals 843, real-time diagnostic images 844 and stored diagnostic images 845. The remote monitoring workstation 804 and the physiology workstation data to afford the operator is the ability to reformat and reposition the various windows presented on each monitor in order to customize the layout and combination of windows presented.

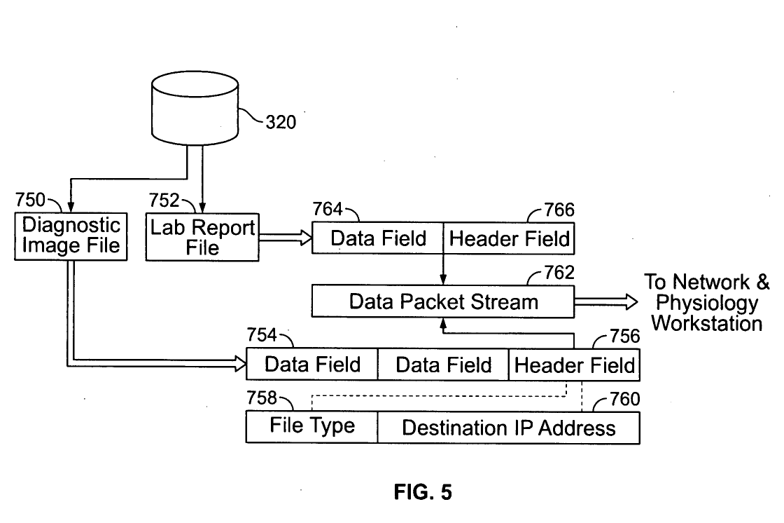

FIG. 5 illustrates a block diagram of a data packet processing sequence for packet sizing patient files and conveying the patient files over the network to the physiology workstation. In the example of FIG. 5, the database 320 is accessed by the server 316 (FIG. 1) to obtain a diagnostic image file 750 and a lab report file 752 associated with a particular patient (identified based on SS number or by a unique medical ID number assigned by medical insurance companies, and the like). The diagnostic image file 750 may represent raw are processed image data, while the lab report file 752 may simply represent a text file or spreadsheet. The server 316 packet sizes the diagnostic image file 750 by importing the image data into data fields 754 and attaching a header field 756 to one or more data fields. In the example of FIG. 5, a common header fields 756 is utilized with multiple data fields 754. Alternatively, a separate header fields 756 may be attached to an associated with each data field 754. The header field includes, among other things, a file type designator 758 and the destination IP address 760 of the physiology workstation 302. Once the diagnostic image file 750 is reformat it into the appropriate packetized protocol, it is conveyed as a data packet stream 762 over the network 300 to the physiology workstation 302.

In the example of FIG. 5, the physiology workstation 302 also requested a lab report file 752 from the database 320. The server 316 obtains and reformats the lab report file 752 from the database 320 into data packets, including at least one data field 764 and at least one header field 766. The data fields 764 include the substantive lab report data, while the header field 766 includes a file type designator and destination IP address. Once packetized, the lab report file 752 is conveyed as a data packet stream to the physiology workstation 302 over the network 300.

Alternatively, the files may be formatted utilizing local area network protocols, wide area network protocols, the TCP/IP protocol, and the like.

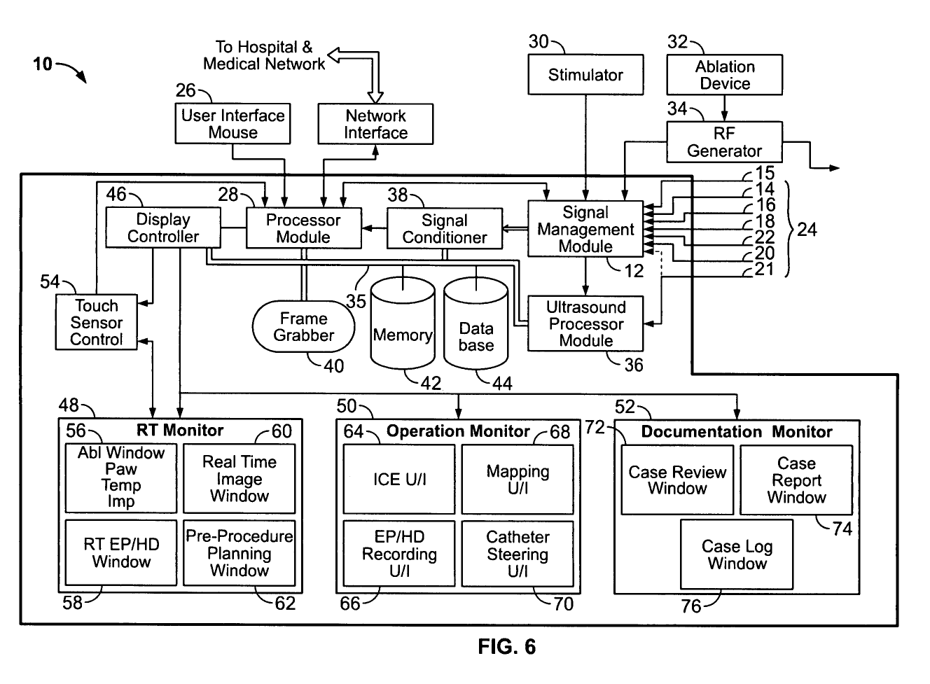

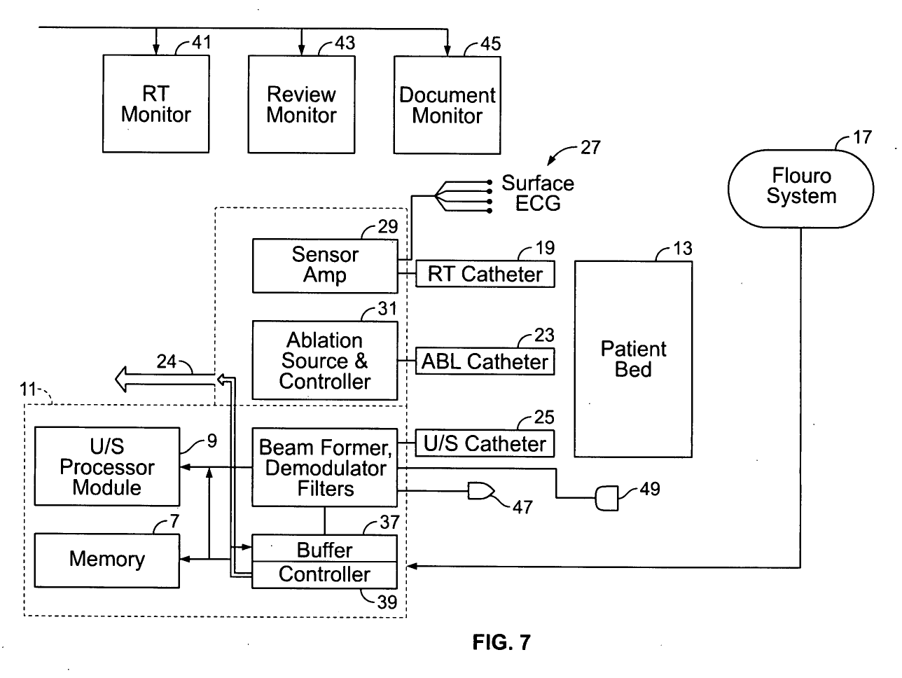

FIG. 6 illustrates a networked physiology workstation 10 in accordance with an embodiment of the present invention. The workstation 10 includes a network interface 8 configured to be joined to the network 300. The network interface is assigned an IP address and operates in the manner discussed above. The workstation 10 may be located in a control room or a procedural room and is utilized in connection with HD, EP and ablation procedures, among other things. FIG. 7 illustrates equipment that is located in a procedure room which may be separate and discrete from the control room (when used) and from a remote monitoring room within the facility (e.g. a hospital, clinic and the like). The workstation 10 is operated by an operator, while the patient and procedure team are located in the procedure room. The workstation 10 integrates, among other things, real-time information, real-time intracardiac echography, fluoroscopic images, mapping data and pre-surgery planning CT & MR images. The workstation 10 offers integrated monitoring and review of HD, EP, patient, and mapping information as well as stored and real-time diagnostic images, ECG signals and IC signals.

As shown in FIG. 7, the procedure room includes an ultrasound system 11, a fluoroscopy system 17 and a patient bed 13 to hold the patient while an HD, EP or ablation procedure is carried out. Fluoroscopy system 17 is provided proximate patient bed 13 to obtain fluoroscopic images of the region of interest while the doctor is conducting a procedure. Catheters 19 (EP or HD), an ablation catheter 23 and ultrasound catheter 25 are provided to be inserted throughout the procedure. Any suitable catheter guidance system, such as a magnetic guidance system, may be used. EP catheter 19 performs sensing and stimulating functions. The ablation catheter 23 may represent an RF ablation catheter, a laser ablation catheter or a cryogenic ablation catheter. The ultrasound catheter 25 is configured to obtain ultrasound images of the region of interest, as well as images that indicate directly the position and placement of catheters and the ablation catheter relative to the region of interest. Alternately, ultrasound catheter 25 may be used to assess tissue motion, luminal size, etc. Surface ECG leads 27 are provided and attached to the patient to obtain surface ECG information. The surface ECG leads 27 and the catheters 19 are joined to a sensor amplifier 29 which amplifies signals sensed by the surface ECG leads 27 and EP catheters 19 prior to transmitting the sensed signals over a communications interface 24. When stimulus pulses are to be delivered to the patient, the stimulus signals are passed either around or through the sensor amplifier 29 to the corresponding catheters 19. An ablation source and controller 31 controls operation of the ablation catheter 23 and provides ablation-related data over the communications interface 24 to the workstation 10 (FIG. 1).

The beamformer 33 is responsible for transmit and receive beam forming operations. The beamformer 33 controls the phase and amplitude of each transmit signal delivered over the link to induce a transmit or firing operation by the ultrasound catheter 25. Reflected echoes are received at the ultrasound catheter 25 and delivered to the beamformer 33 as analog signals representative of the detected echo information at each individual transducer element. Optionally, the beamformer 33 may also control transmission and reception in connection with non-catheter type U/S probes, such as a transesophageal probe 47, a surface cardiac probe 49, an intravenous, intraarterial, intracardiac probes and the like. The beamformer 33 includes a demodulator and filters to demodulate and filter the received analog RF signals and produce therefrom digital base-band I and Q data pairs formed from acquired data samples. The I,Q data pairs are derived from the reflected ultrasound signals from respective focal zones of the transmitted beams. The I,Q data pairs corresponds to each data samples within the region of interest. The beamformer 33 may pass the I,Q data pairs to a FIFO buffer 37 which then passes the I,Q data pairs over the communications interface 24 under the control of the controller 39. Alternatively, the beamformer 33 may directly stream the I,Q data pairs over the communications interface 24 as generated without buffering. Optionally, the beamformer 33 may store the I,Q data pairs in memory 7 in the ultrasound system 11. An ultrasound processor module 9 may be provided in the ultrasound system 11 to process the I, Q data pairs to form ultrasound images that are passed over communications interface 24 and/or stored in memory 7.

A real-time monitor 41, a review monitor 43 and documentation monitor 45 are located proximately the patient bed 13 for viewing by the procedure team and physician during the procedure monitors 41, 43 and 45 and are remotely controlled to present the same information as presented on the real-time monitor 48, operation monitor 50 and documentation monitor 52, respectively, located at the workstation 10.

The workstation 10 includes a signal management module 12 which is configured to receive and transmit a variety of signals and data that are conveyed to and from the patient over leads, cables, catheters and the like. Examples of signals that may be received by the signal management module 12 include intracardiac (IC) signals 14 from EP catheters, patient monitoring signals 15 (e.g., from a blood pressure cuff, SPO2 monitor, temperature monitor, CO2 levels and the like), ECG signals 16 from surface ECG leads 27, pressure signals 18 from an open lumen catheter, and intracardiac signals. The signal management module 12 also receives fluoroscopic imaging data 20 from the fluoroscopic system 17, ultrasound imaging data 21 from the beamformer 33, and ablation data 22 (e.g., power, temperature, impedance) from the ablation source and controller 31. The fluoroscopic system 17 is an x-ray apparatus located in the procedure room. The ultrasound data 21 also may be collected at a transesophageal ultrasound probe, an intraoperative ultrasound probe, a transthoracic probe and/or a cardiac ultrasound probe. Optionally, the ultrasound system 11 may be operated in an acoustic radiation force imaging (ARFI) mode.

The communications interface 24 extends from the workstation 10 to the various equipment located proximate the patient bed. When different rooms are provided the interface 24 extends through the wall or other divider separating the control and procedure rooms, into the procedure room. The communications interface 24 conveys, among other things, IC signals 14, patient monitoring signals 15, surface ECG signals 16, pressure signals 18, fluoroscopic imaging data 20, ultrasound imaging data 21 and ablation data 22. The content and nature of the information conveyed over the communications interface 24 is explained below in more detail. In one embodiment, the communications interface 24 is comprised of physical connections (e.g. analog lines, digital lines, coaxial cables, Ethernet data cables and the like or any combination thereof).

Optionally, the communications interface 24 may include, in whole or in part, a wireless link between the workstation 10 in the control room and one or more of the ultrasound, fluoroscopic, ablation, and EP instruments, devices, apparatus and systems in the procedure room 11. For example, ultrasound data 21 may be communicated wirelessly from a transmitter that is located within the procedure room 11 at the beamformer 33 to a receiver that communicates with the workstation 10 in the control room. The receiver would then convey the imaging data 21 to the signal management module 12.

The signal management module 12 selectively controls access of signals and data onto the communications interface 24. The signal management module 12 may comprise a simple configuration of switches that are manually operated by the user via the user interface 26. Alternatively, switches in the signal management module 12 may be automatically controlled by the processor 28 based upon various criteria including, among other things, the type of procedure currently being conducted. The signal management module 12 may include processing capabilities (e.g. a CPU, DSP and the like) to internally and automatically decide certain switching operations. The signal management module 12 may include memory, such as to temporarily buffer incoming and/or outgoing signals and/or data from/to the communications interface 24. The communications interface 24 conveys analog and digital signals. In the event that the communications interface 24 conveys analog signals, the signal management module 12 may include analog to digital converters to convert the analog signals to digital data and vise versa.

The signal management module 12 may communicate directly with an external stimulator 30. The stimulator 30 may deliver electrical signals (such as for pacing) directly over interface 24, or through the signal management module 12 and the IC leads 14, to one or more catheters 19 positioned within the patient. Examples of stimulators are the Micropace by Micropace Pty Ltd and the Bloom offered by Fisher Imaging. Optionally, the signal management module 12 may process or otherwise interact with the signals to/from the leads 14 and catheters 19. The signal management module 12 may receive the signals from the leads 14, catheters 19 and otherwise, digitize and process such signals, store the signals in internal memory and send on the signals. The pacing signal may or may not go through the signal management module 12, and may not go through the amplifier.

The workstation 10 is used in an EP study to provide a detailed evaluation of the hearts electrical system. During an EP study, typically 3-5 catheters 19 are used. Each EP catheter 19 includes platinum electrodes spaced near the tip of the catheter, where such electrodes have the ability to record electrical signals from inside the heart as well as deliver stimulus pulses to the heart from different locations, such as to pace the heart. The workstation 10 evaluates normal and abnormal conductions and rhythms. The protocol used during the EP study may vary from site to site or procedure to procedure (e.g. corrected sinus node recovery time, AV Wenckebach and the like).

The incoming signals from the patient over the communications interface 24 are passed from the signal management module 12 to a signal conditioning circuit 38 which performs various signal processing operations upon the incoming signals. The signal conditioning circuit 38 passes conditioned signals to the processor module 28 and optionally may pass the conditioned signals to a frame grabber 40 or directly to memory 42 or a database 44. The processor module 28 manages overall control and operation of the workstation 10. The processor module 28 receives user inputs through the user interface 26. The processor module 28 stores data, images and other information in the memory 42 and/or in the database 44. The frame grabber 40 also accesses memory 42 and database 44 in order to obtain and store various data, images and the like. While the memory 42 and database 44 are shown as part of the workstation 10, it is understood that one or both of the memory 42 and database 44 may be part of the workstation 10, separate from, but located locally to the workstation 10 (e.g. in the control room) or remote from the workstation 10 and the control room (e.g. in another part of the facility or at an entirely separate geographic location (e.g. a different hospital, university, state, country and the like)).

The memory 42 and database 44 may store diagnostic images, such as CT and MR images acquired prior to the procedure, and ultrasound images acquired prior to, during, or after the procedure. The stored images facilitate pre- and post-procedure analysis for image optimization, manipulation and analysis. The ultrasound images may represent intracardiac ultrasound images obtained from the ultrasound catheter 25. Optionally, the ultrasound images may be obtained utilizing a transesophageal probe 47, an interoperative probe, and an external cardiac probe 49. The ultrasound images, measurements, and/or other data obtained can be stored locally to ultrasound system 11 or via network 300 on physiology workstation 10 and/or server 316.

In each of the workstation 10 and U/S system 11, the timing information may be derived from the time of day, or from a reference clock. Alternatively, the various processors may have synchronized clocks which result in all the various systems being synchronized to the identical spot in the cardiac cycle. Alternatively, the timing information may be associated with the cardiac cycle of the patient which is determined by ECG or EP signals.

The processor module 28 communicates uni-directionally or bi-directionally with the display controller 46 which controls monitors 48, 50 and 52. The monitors 48, 50 and 52 may simply present displayed information as explained hereafter. Optionally, the monitors 48, 50 and 52 may include input buttons for operation by the user to directly enter certain commands and instructions at the monitor 48, 50 and 52. Optionally, the monitors 48, 50 and 52 may represent touch sensitive screens that enable the user to enter information directly by touching active areas of a corresponding monitor 48, 50 and 52.

In the example of FIG. 6, a touch sensor control 54 is illustrated that detects touch actions relative to monitor 48. The touch sensor control 54 provides the results of the touch action to the processor 28. The touch action result may simply represent an X,Y coordinate at which a touch event occurred. Alternatively, the touch sensor 54 may first determine the X,Y coordinate of the touch event and subsequently determine the intended action or instruction based upon the display content of monitor 48 under the control of the display controller 46. For example, the touch sensor control may return a “select drop down menu”.

In the example of FIG. 6, monitors 48-52 have been assigned different categories of functions (e.g. real-time monitoring, operations monitoring, documentation monitoring and the like). Monitor 48 presents numerous windows, such as ablation window 56, a real-time EP monitoring window 58, a real-time image window 60 and a preprocessing planning window 62. The monitor 50 displays windows related to operation control, such as an ICE user interface window 64, an EP/HD recording user interface window 66, a mapping user interface window 68 and a catheter steering user interface window 70. The user interface windows 64-70 allow the operator to enter and change parameters, modes, patient information, values and the like in connection with a particular EP study. User interfaces 64, 66, 68, and 70 allow control of ultrasound system 11 to perform such functions as change modes, alter the angle of view, increase or decrease depth of view, etc. These functions may be accomplished either via soft keys (such as those produced by a visual keyboard simulator or software module) or via a remote keyboard and mouse or a combination of these. Soft keys refers to the graphical representation on a monitor of buttons or knobs which are part of ultrasound system 11. These softkeys may be activated either via keyboard and/or by mouse. Monitor 52 is configured to present windows associated with documentation of a particular patient case. Monitor 52 presents a case review window 72, a case reporting window 74 and a case log window 76. The case-related windows 72-76 allow the user to review patient history information, as well as current patient information associated with the EP or HD study. The monitors 48, 50 and 52 may also present prerecorded patient information, such as stored physiology signals, stored patient logs, stored diagnostic images, stored lab work, prior physician reports, patient demographics and the like.

The workstation 10 integrates the display of ultrasound images with other EP or HD study information and/or ablation procedure information by utilizing one or more of monitors 48, 50 and 52. For example, real-time image window 60 may present ultrasound images obtained from an ultrasound catheter, while planning window 62 presents previously acquired CT or MR images. Integrating the ultrasound images into the workstation affords, among other things, an improved standard of care, increased user confidence and shorter procedure time.

Optionally, the real-time image window 60 may present ultrasound images as a loop, in which a sequence of ultrasound frames is acquired and associated with one or more cardiac cycles. The loop of ultrasound images may be repeatedly displayed or frozen. Control of ultrasound functions may be accomplished via ultrasound system 11 itself or via a remote keyboard, mouse and softkeys or any combination of these. While the real-time image window 60 presents the ultrasound images, the real-time EP/HD window 58 simultaneously displays real-time EP signals corresponding to the ultrasound cine loop. Optionally, one screen may be static while the other screen updates with live images, where the user select which screen is live. The planning window 62 may present associated mapping data acquired earlier during the EP or HD study.

The signal management module 12 also communicates directly with an ablation control device 32 which is used to control various ablation procedures. The ablation control device 32 may constitute RF catheter ablation, laser catheter ablation, cryogenic ablation and the like. The ablation device 32 is attached to a generator 34 that produces the energy utilized to achieve ablation. Optionally, the ablation device may be a single module or unit that both controls and delivers the energy. For example, in an RF ablation, cryoablation, or laser ablation system, the generator 34 represents a RF generator, cryoablation generator or a laser source. During RF catheter ablation, energy is delivered from a RF generator through an RF catheter having a tip located proximate anatomy that is desired to undergo ablation. Ablation is generally performed in order to locally destroy tissue deemed responsible for inducing an arrhythmia. The RF energy represents a low-voltage high-frequency form of electrical energy that produces small, homogeneous, lesions approximately 5-7 millimeters in diameter and 3-5 millimeters in depth.

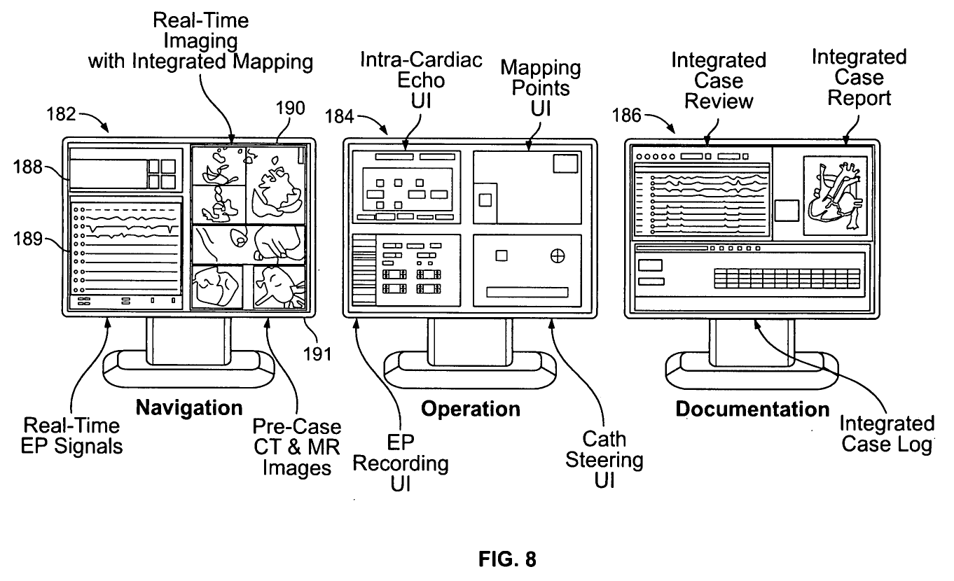

FIG. 8 illustrates more detailed examples of the window content that may be presented in various combinations on the monitors 41, 43, 45, and 48-52. The monitors in FIG. 8 represent a navigation monitor 182, an operations monitor 184 and a documentation monitor 186. The navigation monitor includes an ablation window 188, real-time EP signal window 189, real-time imaging window 190 with integrated mapping indicia and pre-case image window 191 (e.g. previously acquired CTR MR images). Optionally, any of the monitors may provide the real time imaging with integrated mapping. The operations monitor 184 includes windows associated with intracardiac echography, mapping, catheter steering and EP recording. The documentation monitor 186 includes windows associated with integrated case review, integrated case reports and an integrated case log. The monitors 182, 184 and 186 may also present prerecorded patient information, such as patient demographic information, interventional medical procedure history, prior physician/lab reports, past measured physiologic performance, diagnostic image information, and prior physiology studies.

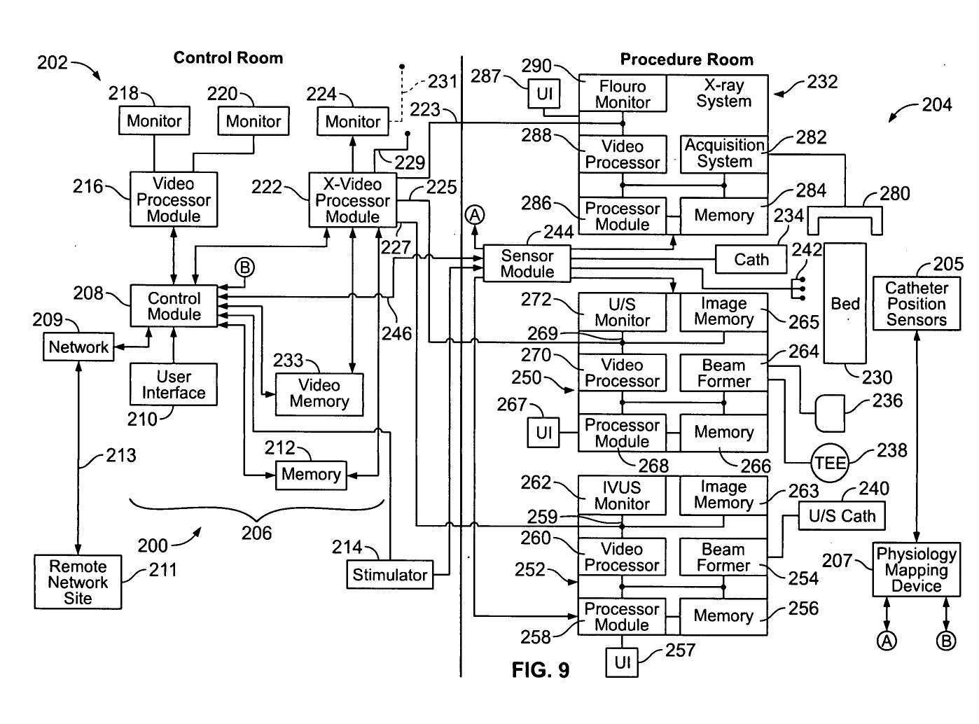

FIG. 9 illustrates a networked image management system 200 formed in accordance with an embodiment of the present invention. The image management system 200 may be distributed between a control room 202 and procedure room 204 or, alternatively, may be all located in the procedure room 204. Thus the image management system 200 may be located entirely in the procedure room 204. A physiology workstation 206 (e.g., EP or HD workstation) is provided to control and coordinate EP or HD procedures, ablation procedures and the like. The physiology workstation 206 includes a control module 208 that is controlled by an operator through user interface 210. The control module 208 includes network interface joining the system 200 to the network 209 and a remote network site 211. By way of example, the network 209 may resemble the network 300 of FIG. 1 and the remote network site 211 may represent the server 316 or one or more of the workstations, computers and hand-held devices discussed above.

Memory 212 stores various information explained below in more detail. A stimulator 214 is provided to generate stimulus signals delivered to the patient in the procedure room 204. A physiology video processor module 216 communicates with the control module 208 and controls monitors 218 and 220. An external video processor module 222 is also provided within the workstation 206. The external video processor module 222 communicates with control module 208 and controls a real-time imaging monitor 224. Optionally, the physiology and external video processor modules may be combined as a single module and/or may implemented utilizing a single or parallel processors.

A physiology mapping device 207 is provided in the procedure room 204 and is joined to the workstation 206 over link B and to the sensor module 244 over link A. The physiology mapping device 207 communicates with catheter position sensors 205 to monitor the position of EP, HD and/or mapping catheters, while being positioned within the heart. The workstation 206 integrates, among other things, real-time EP and HD information, real-time intracardiac (IC) echography, transesophageal ultrasound, transthoracic ultrasound, fluoroscopic images, EP mapping data and pre-surgery planning CT & MR images. The workstation 206 offers integrated monitoring and review of EP, HD, patient, and mapping information as well as stored and real-time diagnostic images, ECG signals, ultrasound images in still or sequence format and IC signals.

The procedure room 204 includes a patient bed 214 to hold the patient during pre-procedure intracardiac mapping and during EP, HD and ablation procedures. A fluoroscopy system 232 is provided proximate patient bed 214 to obtain fluoroscopic images of the region of interest while the doctor is conducting mapping or a procedure. EP or HD catheters 234, ultrasound probes 236, 238 and an ultrasound probe 240 are provided for use throughout the procedure. The ultrasound catheter 240 and ultrasound probes 236, 238 are configured to obtain ultrasound images of the region of interest, as well as images that indicate directly the position and placement of other instruments, devices and catheters, such as a defibrillator or pacemaker lead, catheter 234, an ablation catheter and the like relative to the region of interest. Surface ECG leads 212 are provided and attached to the patient to obtain surface ECG information.

An ultrasound system 250 and an intravascular ultrasound (IVUS) catheter or intracardiac echo catheter (ICE) 252 are joined to, and control, the ultrasound probes 236, 238 and catheter 240. The ultrasound catheter 240 may generally represent an intravascular ultrasound (IVUS) catheter, in that the catheter 240 and IVUS system 252 may be used to perform diagnostic ultrasound examination of any and all portions of a subjects vascular structure, peripheral veins, peripheral arteries and the like. Ultrasound catheter 240 may likewise be an intracardiac echo (ICE) catheter, which may be used to perform diagnostic examinations of cardiac structures and function, including visualization of the pulmonary veins, visualization of the anatomic response to ablation, assessment of aberrant connections between various chambers of the heart and the like. A user interface 257 permits an operator to control operation of the IVUS or ICE system 252, and to enter modes, parameters and settings for the IVUS or ICE system 252. The IVUS or ICE system 252 includes a beamformer 254 that is responsible for transmit and receive beamforming operations. The beamformer 254 controls the phase and amplitude of each transmit signal delivered over the link to induce transmit or firing operations by the ultrasound catheter 240.

The beamformer 254 may include a demodulator and filter (or a processor programmed) to demodulate and filter the received echo signals. The beamformer 254 generates RF signals from echo signals and performs RF processing to produce digital base-band I and Q data pairs formed from the RF signals associated with acquired data samples. An I,Q data pair corresponds to each data sample within the region of interest. The beamformer 254 may pass the I,Q data pairs to memory 256, or directly to processor module 258.

The I,Q data pairs are processed by mode-related modules (e.g., B-mode, color Doppler, power Doppler, M-mode, spectral Doppler anatomical M-mode, strain, strain rate, and the like) of the processor module 258 to form 2D or 3D data sets of image frames, volumetric data sets and the like. The image frames are stored in memory 256. The processor module 258 may record, with each image frame, timing information indicating a time at which the image frame was acquired. The processor module 258 may also include a scan conversion module to perform scan conversion operations to convert the image frames from Polar to Cartesian coordinates. A video processor module 260 reads the image frames from memory 256 and displays the image frames on the ultrasound monitor 262 in real time during the procedure is being carried out on the patient. Optionally, the video processor module 260 may store the image frames in an image memory 263, from which the images are read and displayed on IVUS monitor 262. These image frames can then be sent either to the study, or, at the end of the case, to the physicological workstation 206 and/or server 316.

A video link 259 is maintained between the video processor 260, image memory 263 and ultrasound monitor 262. The ultrasound system 252 includes a video output (e.g., a VGA output) that is connected to a video link 227 (e.g., a VGA cable). The ultrasound system 250 includes a transmitter (within beamformer 264) which drives ultrasound probes 236, 238. A user interface 267 permits an operator to control the operation of, and enter modes, parameters and settings for, the ultrasound (U/S) system 250. The beamformer 264 processes the signals for steering, focusing, amplification, and the like. The beamformer 264 also filters and demodulates the RF signals to form in-phase and quadrature (I/Q) data pairs representative of the echo signals from data samples. The RF or I/Q signal data may then be routed to the memory 266 for storage or directly to the processor module 268. The processor module 268 acquires ultrasound information (i.e., the RF signal data or IQ data pairs) from memory 266 and prepares frames of ultrasound information (e.g., graphical images) for storage or display. The processor module 268 provides the ultrasound information to the video processor 270. The video processor 270 stores image frame data in the image memory 265 and outputs the video signals that drive the monitor 272. A video link 269 is maintained between video processor module 270, image memory 265 and U/S monitor 272. The video link 225 conveys to the physiology workstation 206 the identical video signals as presented to the U/S monitor 272.

The processor module 258 in the ultrasound system 250 may also receive hemodynamic, inter-cardiac and/or surface ECG signals from the sensor module 244, surface leads 242 and catheter 234. Optionally, the processor modules 258 and 268 may receive respiratory signals corresponding to the breathing cycle of the patient. The processor modules 258 and 268 utilize the IC signals, HD signals, ECG signals and/or respiratory signals to derive timing information that is tagged to each ultrasound image frame generated by the scanned converter 326 (FIG. 2). In one mode of operation, the ultrasound system 250 displays sequences of images captured by the probes 236, 238. One or more of the images may be displayed in synchronism with an event trigger determined by in the processor module 268. Optionally, the IVUS/ICE catheters 252 and/or the ultrasound system 250 may be operated in an acoustic radiation force imaging (ARFI) mode.

The procedure room 204 may include various equipment and systems, such as an x-ray system 232 that controls a rotating support arm 280. The modes, parameters and other settings of the x-ray system 232 are entered and controlled from the user interface 287. The support arm 280 includes a x-ray source and a x-ray detector on opposite ends thereof. The x-ray detector may represent an image intensifier, a flat panel detector, a change coupled device and the like. The x-ray detector provides fluoroscopy data to a data acquisition system 282 which stores the x-ray data in memory 284. A processor module 286 processes the x-ray data to generate x-ray images that may be stored in memory 284 or passed directly to video processor module 288.

In each of the x-ray system 232, IVUS/ICE catheters 252 and U/S system 250, the timing information may be derived from the time of day, or from a reference clock. Alternatively, the various processors may have synchronized clocks which result in all the various systems being synchronized to the identical spot in the cardiac cycle. Alternatively, the timing information may be associated with the cardiac cycle of the patient which is determined by the EP signals provided from the sensor module 244.

The workstation 206 includes a physiology control module 208 which is configured to receive and transmit a variety of signals and data that are conveyed to and from the patient over leads, cables, catheters and the like. Examples of signals that may be received by the control module 208 include intercardiac (IC) signals and/or hemodynamic signals from catheters 234, patient monitoring signals (e.g., from a blood pressure cuff, SPO2 monitor, temperature monitor, CO2 levels and the like), ultrasound images such as Doppler, ECG signals from surface ECG leads 212. The control module 208 manages overall control and operation of the workstation 206. The EP control module 208 receives user inputs through the user interface 210. The EP control module 208 stores data, images and other information in the memory 212. The EP video processor module 216 accesses memory 212 in order to obtain and store various data, signal traces, images and the like. The memory 212 may store diagnostic images, such as ultrasound CT and MR images acquired prior to the procedure. The stored images facilitate pre- and post-procedure analysis for image optimization, manipulation and analysis. The control module 208 communicates uni-directionally or bi-directionally with video processor module 216 which controls monitors 218 and 220. The monitors 218 and 220 may simply present displayed information as explained hereafter. Optionally, the monitors 218 and 220 may include input buttons for operation by the user to directly enter certain commands and instructions at the monitor 218 and 220. Optionally, the monitors 218 and 220 may represent touch sensitive screens that enable the user to enter information directly by touching active areas of a corresponding monitor 218 and 220. The touch sensitive screens can be operated under control of a visual keyboard simulator software module and used to control ultrasound system 250.

The workstation 206 integrates the display of real-time ultrasound and fluoroscopy images with other EP/HD study information and/or ablation procedure information by utilizing one or more of monitors 218, 220 and 224. For example, the real-time image monitor 224 may present ultrasound images obtained from an ultrasound catheter, while the planning window presents previously acquired CT or MR images. Control of the ultrasound system via the physiology workstation allows a single user to have access to and control the gathering of various types of data including intracardiac signals, hemodynamic waveforms, vital signs etc. Integrating the ultrasound images into the workstation affords, among other things, an improved standard of care, increased user confidence and shorter procedure time.

The real-time image monitor 224 may present ultrasound images as a cine loop, in which a sequence of ultrasound frames is acquired and associated with one or more cardiac cycles. The cine loop of ultrasound images may be repeatedly displayed or frozen. While the real-time image monitor 224 presents the ultrasound Optionally, the various images may be displayed on any of the screens images, the monitor 218 simultaneously displays real-time EP or HD signals corresponding to the ultrasound cine loop. The workstation 206 includes an external video processor module 222 that has access to memory 212 and communicates with the control module 208. The external video processor module 222 controls a separate monitor 224 provided as part of the workstation 206. Monitor 224 is positioned immediately adjacent monitors 218 and 220 in order that all 3 monitors may be reviewed simultaneously by an operator of the workstation 206, while the ultrasound system is remotely controlled.

The external video processor module 222 receives video input signals 223, 225, and 227 from the x-ray system 232, the ultrasound system 250 and the IVUS system 252, respectively. The video signals 223, 225 and 227 are directly attached to the video signals used to drive the fluoroscopy monitor 290, ultrasound monitor 272, and IVUS monitor 262, respectively. The external video processor module 222, under direction of the control module 208, affords a comprehensive image management system under which fluoroscopy and ultrasound images may be viewed in real-time at the workstation 206. The external video processor module 222 includes additional video input signals (e.g., such as signal 229) from any standard video source.

In at least one embodiment, monitoring workstations are provided remote from the physiology workstation. The monitoring workstation co-displays the same information as the physiology workstation and permits an operator of the monitoring workstation to update patient information, patient logs and the like during the procedure. The physiology network stores the new physiology study and case log in the patient database, along with any updates entered at monitoring workstations. The information displayed at the physiology workstation may also be displayed real-time on any personal computer, personal digital assistant, cell phone and the like joined to the network. For instance, computers located in individual doctors offices, or in an administrative office may be utilized to view and, based upon network privileges or permissions, may update the patient information during the study. The physiology workstation, monitoring workstations and office computers support “same time” text and/or audio communication with one another, such as to support remote consultations and the like.

In some embodiments the physiology workstation is connected either via direct connection to an ultrasound system utilizing fiber optic or standard networking cabling allowing bidirectional communication between the two systems using standard protocols. This connection allows remote control of the ultrasound system via the user interface of the physiology workstation. Ultrasound functions such as changing modes, changing gain, measurements, storing of images, etc can be controlled via the physiology workstation. In addition the clocks of the two systems are synchronized allowing the user to know that data points that occur at discreet points in time represent data collected simultaneously. Images and measurements may be stored to the physiology workstation and displayed to the user concomitant with other data obtained by the physiology workstation.

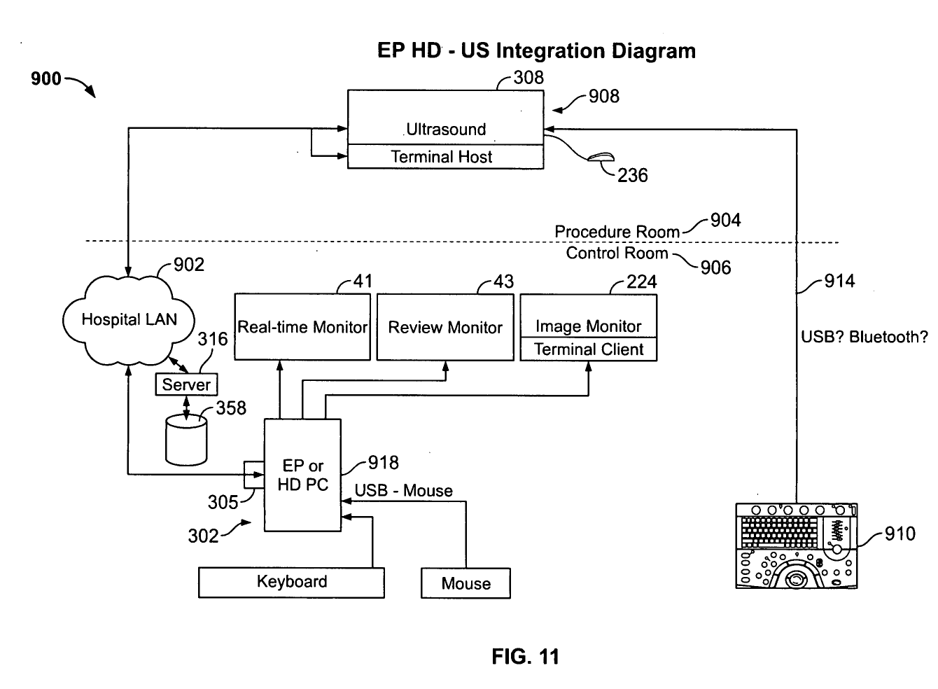

More particularly, and referring to FIG. 11, in some configurations of the present invention, a physiological network 900 is provided that is configured to operate with a medical network 902. An ultrasound system 308 is located in a procedure room 904. Ultrasound system 308 includes an ultrasound probe 236. A physiological workstation 302 (also referred to herein as a “local workstation”) is configured to operate in a procedure room 906 and is operatively coupled via medical network 902 to display ultrasound signals obtained from a subject during an ultrasound procedure carried out on the subject. Local workstation 302 has a network interface 305 configured to communicatively couple to medical network 902. A database 358 storing patient records associated with the subject undergoing the physiological procedure is also provided. A server 316 is operatively coupled to medical network 902 and database 358. Server 316 is configured to provide, to a local workstation 309 (which can be a display on ultrasound system 308) and remote workstation 302, a patient record associated with the subject. Local workstation 309 co-displays the ultrasound signals and information from the patient record to an operator of local workstation 309. A remote workstation 302 is configured to operate in a control room 906 different from procedure room 904, so that a person in the control room can control ultrasound system 308 while receiving, processing, and displaying the ultrasound signals obtained from the subject in real-time 41, while an ultrasound procedure is being performed on the subject. Remote workstation 302 can comprise an EP workstation, an HD workstation, or a combination EP/HD workstation. Ultrasound probe 236 can be, for example, an intravascular ultrasound probe, an intracardiac probe, and ultrasound system 308 can be, for example, a 2-D ultrasound system or a 3-D ultrasound system. In some configurations, remote workstation 302 and either or both local workstation 908 or ultrasound system 308 have synchronized clocks. These clocks (which may comprise embedded software or firmware modules) can be synchronized, for example, to the time on server 316.

In some configurations of the present invention, a keyboard 910 is provided in control room 906. Keyboard 910 is configured to communicate with ultrasound system 308 via either a wired connection 912 separate from medical network 902 or a wireless connection 914 (see FIG. 11) separate from medical network 902. Wired connection 912 can be, for example, a custom cable or a USB connection. Wireless connection 914 can be, for example, any of the 802.11 wireless protocol connections or a bluetooth connection.

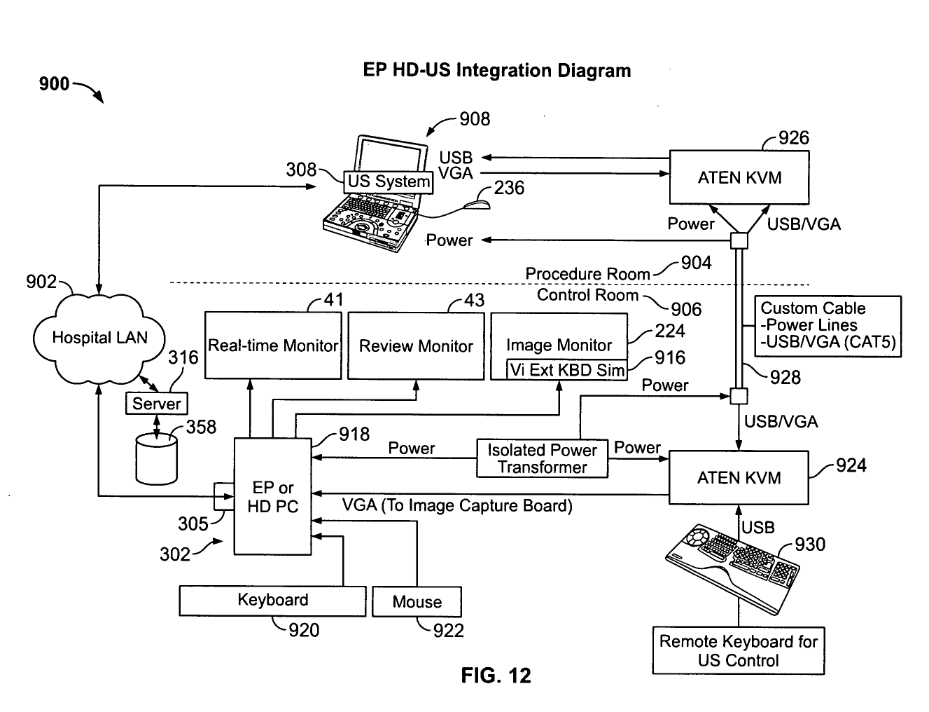

Also, in some configurations of the present invention and referring to FIG. 12, remote workstation 302 includes an image monitor 224 and a visual keyboard simulator software module configured to run, at least in part, on remote workstation 302. (To receive simulated keypresses, a portion of the keyboard simulator software module may be configured to run on local workstation 908 in some configurations.) An image 916 of a keyboard is displayed on image monitor 224. Image monitor 224 may include a touchscreen for operating the keyboard simulator from image 916, or EP or HD PC 918 may be configured to activate simulated keypresses on image 916 using a separate physical keyboard 920 or mouse 922. Virtual keypresses from keyboard 916 are transferred through medical network 902. In some configurations, a pair of keyboard/video/mouse (KVM) switches 924, 926 and a custom cable 928 are provided to communicate between a remote keyboard 930 and ultrasound system 308. However, because some KVM switches 924, 926 are unable to effectively communicate signals that control ultrasound system 308. Therefore, signals that control ultrasound system 308 are generated by the visual keyboard simulator software module in response to simulated keypresses and transmitted via LAN 902 to ultrasound system 308.

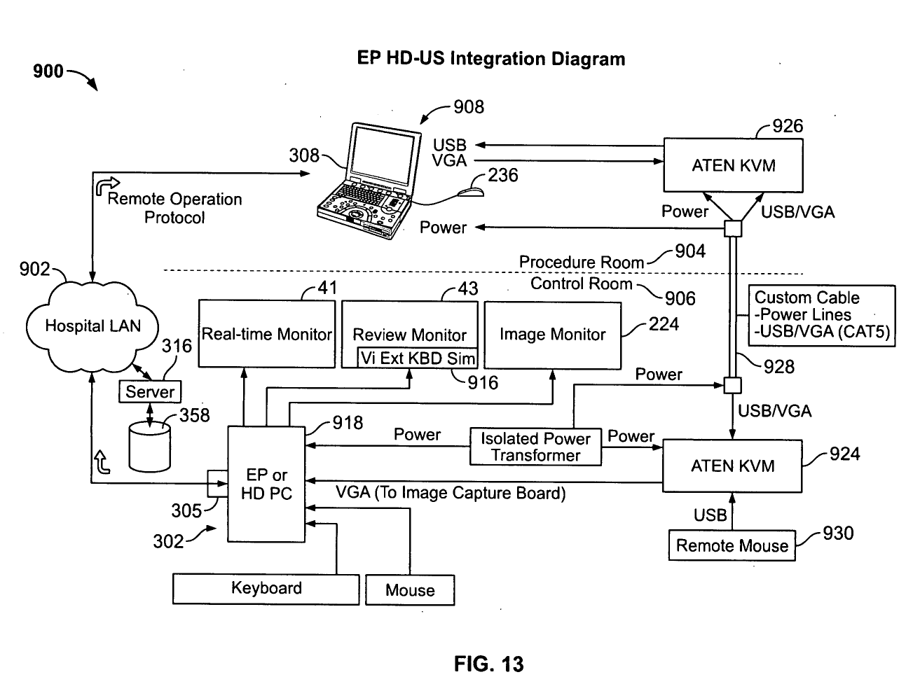

In some configurations and referring to FIG. 13, the keyboard simulator software module is configured to display keyboard image 916 on review monitor 43 instead of, or in addition to, image monitor 224. Review monitor 43 may comprise a touchscreen.

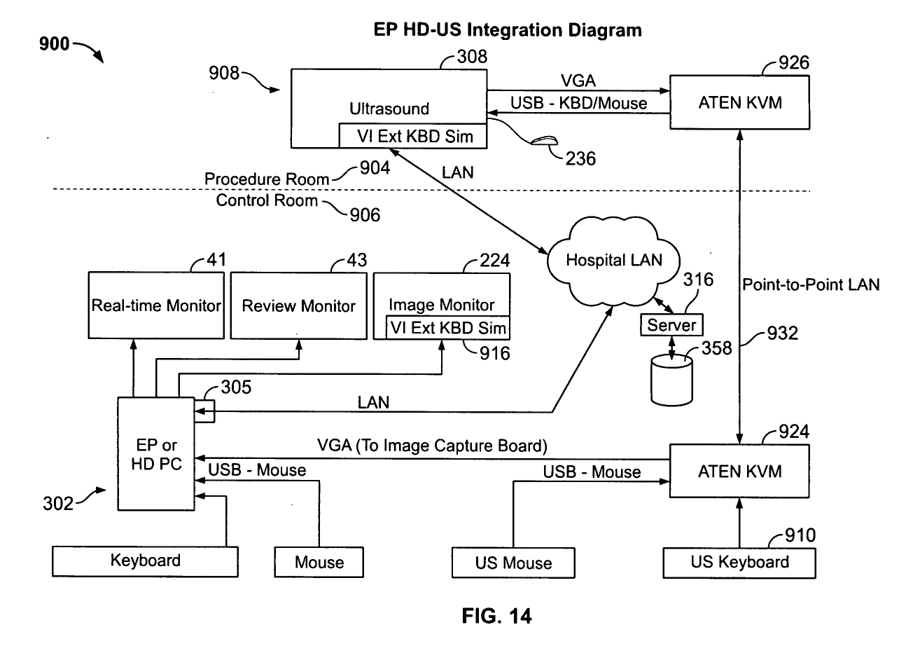

In yet another configuration or configurations and referring to FIG. 14, a pair of KVM switches 924, 926 is provided and a point-to-point wired or wireless local area network (LAN) 932 configured to communicatively couple local workstation 908 to remote workstation 302 is also provided. Data communicated via KVM switches 924, 926 and LAN 932 exclude control signals resulting from use of the visual keyboard simulator software module for controlling ultrasound system 308. Such control signals are instead communicated, for example, via medical network 902.

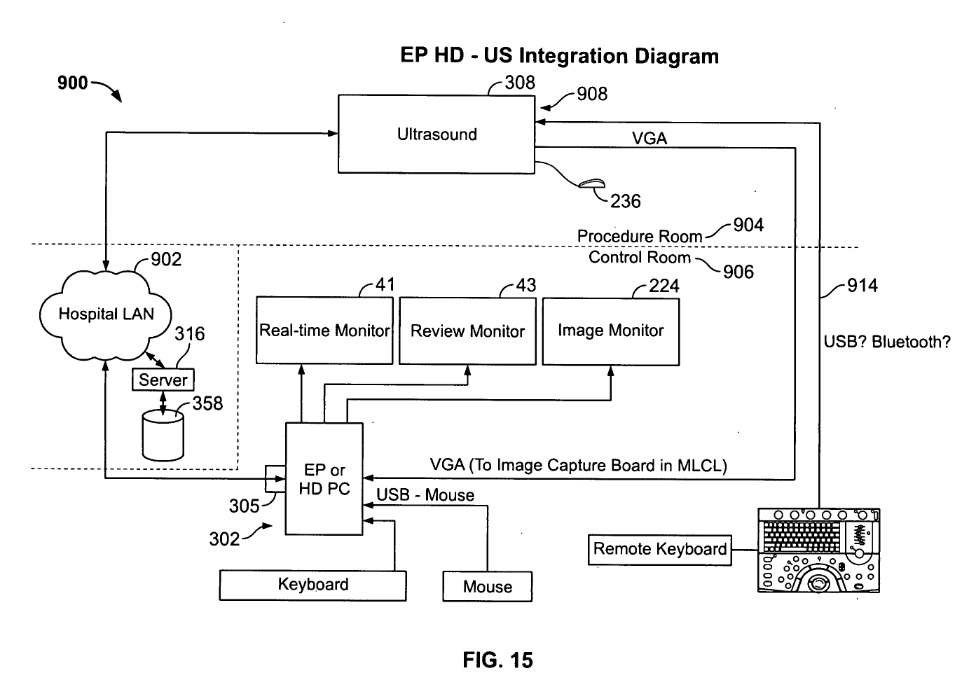

In some configurations and referring to FIG. 15, a keyboard 910 in control room 906 is configured to communicate with ultrasound system 308 via a connection 914 separate from medical network 902. Connection 914 is either (or both) a wired connection (such as a USB connection) separate from medical network 902 or a wireless connection (e.g., bluetooth, 802.11 wireless) separate from the medical network 902.

In all of the above configurations, keyboard 910 can be a keyboard that provides all or essentially all of the keys that are present on ultrasound system 308. The use of such a keyboard (in configurations that do not exclude physical keyboard control of ultrasound system 308) allow all or essentially all of the functions of ultrasound system 308 to be performed remotely by the same keypress or keypresses that would be performed locally. However, keyboard 910 can replaced with a standard PC keyboard 930 if the necessary ultrasound control functions are mapped to the available keys on PC keyboard 930. Other types of keyboards may also be used with appropriate mappings.

Unless otherwise explicitly excluded, in configurations in which a keyboard is used, a mouse or other suitable pointing device and/or a voice recognition module and microphone may also be provided in conjunction with, or in appropriate cases, instead of the keyboard.

The term “co-displays” is not limited to displaying information on a common CRT or monitor, but instead refers also to the use of multiple monitors located in immediately adjacent one another to facilitate substantially simultaneous viewing by a single individual.

The figures illustrate diagrams of the functional blocks of various. The functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like). Similarly, the programs may be stand alone programs, may be incorporated as subroutines in an operating system, may be functions in an installed imaging software package, and the like.

While the invention has been described in terms of various specific embodiments, those skilled in the art will recognize that the invention can be practiced with modification within the spirit and scope of the claims.

What is claimed is:

1. A physiology network configured to operate with a medical network, said physiology network comprising:
an ultrasound system including an ultrasound probe;
a local workstation configured to operate in a procedure room with the ultrasound system and operatively coupled to the ultrasound system to display ultrasound signals obtained from a subject during an ultrasound procedure carried out on the subject, said local workstation having a network interface configured to communicatively couple to the medical network;
a database storing patient records associated with the subject undergoing the physiology procedure;
a server, operatively coupled to the medical network and the database, for managing and controlling access to the database, the server configured to provide, to the local workstation and to a remote workstation, a patient record associated with the subject, the local workstation co-displaying the ultrasound signals and information from the patient record to an operator of the local workstation; and
a remote workstation configured to operate in a control room different from the procedure room and configured to operate the ultrasound system remotely, so that a person in the control room can control the ultrasound system while receiving, processing, and displaying the ultrasound signals obtained from the subject in real time, while an ultrasound procedure is being performed on the subject;
said remote workstation comprising an EP workstation, an HD workstation, or a combination EP/HD workstation.

2. A physiology network in accordance with claim 1, wherein the ultrasound probe is an intravascular ultrasound probe.

3. A physiology network in accordance with claim 1, wherein the ultrasound system is a 3-D ultrasound system.

4. A physiology network in accordance with claim 1 wherein the remote workstation and either or both the local workstation or the ultrasound system have synchronized clocks.

5. A physiology network in accordance with claim 1, further comprising a keyboard in the control room configured to communicate with said ultrasound system via at least one of a wired connection separate from medical network or a wireless connection separate from the medical network.

6. A physiology network in accordance with claim 1 wherein the remote workstation comprises an image monitor and a visual keyboard simulator software module configured to run, at least in part, on the remote workstation, to display on the image monitor, and to communicate with the local workstation to thereby control the ultrasound system.

7. A physiology network in accordance with claim 6 further comprising a pair of keyboard/video/mouse (KVM) switches and a point-to-point local area network (LAN) configured to communicatively couple the local workstation to the remote workstation, wherein data communicated via said KVM switches and said LAN exclude control signals resulting from use of said visual keyboard simulator software module to control the ultrasound system.

8. A physiology network in accordance with claim 1 wherein the remote workstation comprises a review monitor, the remote workstation comprises a visual keyboard simulator software module configured to run, at least in part, on the remote workstation, to display on the review monitor, and to communicate with the local workstation to thereby control the ultrasound system.

9. A physiology network in accordance with claim 8 further comprising a pair of keyboard/video/mouse (KVM) switches and a point-to-point local area network (LAN) configured to communicatively couple the local workstation to the remote workstation, wherein data communicated via said KVM switches and said LAN exclude control signals resulting from use of said visual keyboard simulator software module to control the ultrasound system.

10. A physiology network in accordance with claim 1 wherein the remote workstation comprises a remote physical keyboard, a pair of keyboard/video/mouse (KVM) switches, and a point-to-point wired or wireless connection, said point-to-point wired or wireless connection and said KVM switches configured to communicatively couple the remote physical keyboard to the remote workstation to thereby control the ultrasound system.

11. A method for managing and distributing patient and physiology information over a network joined to a database, the method comprising:
using a local workstation and an associated ultrasound system in a procedure room during a physiology procedure to obtain physiology signals from the subject, wherein the physiology signals include ultrasound signals;
using a remote workstation in a control room different from the procedure room to operate the ultrasound system remotely, so that a person in the control room can control the ultrasound system while receiving, processing, and displaying the ultrasound signals obtained from the subject in real time, while an ultrasound procedure is being performed on the subject;
processing the physiology signals at the remote workstation in real-time during the physiology study;
requesting from the database a pre-recorded patient record associated with the subject, the pre-recorded patient record being generated and stored prior to the physiology procedure;
accessing the database to obtain the pre-recorded patient record associated with the subject; and
providing, to the physiology workstation, the patient record associated with the subject.

12. A method in accordance with claim 11, wherein the ultrasound probe is an intravascular ultrasound probe.

13. A method in accordance with claim 11, wherein the ultrasound system is a 3-D ultrasound system.

14. A method in accordance with claim 11, further comprising synchronizing the remote workstation and either or both the local workstation or the ultrasound system.

14. A method in accordance with claim 11, wherein the remote workstation comprises a keyboard, and the method further includes communicating with the ultrasound system via at least one of a wired connection separate from medical network or a wireless connection separate from the medical network.

15. A method in accordance with claim 11 wherein the remote workstation comprises an image monitor, the remote workstation comprises a visual keyboard simulator software module, and the method further comprises running at least a portion of the keyboard simulator on the remote workstation, displaying a simulated keyboard on the image monitor, and using the keyboard simulator to communicate with the local workstation to thereby control the ultrasound system.

16. A method in accordance with claim 15 further comprising using a pair of keyboard/video/mouse (KVM) switches and a point-to-point local area network (LAN) to communicatively couple the local workstation to the remote workstation, and excluding control signals resulting from use of said visual keyboard simulator software module to control the ultrasound system from data communicated via said KVM switches and said LAN.

17. A method in accordance with claim 11 wherein the remote workstation comprises a review monitor, the remote workstation comprises a visual keyboard simulator software module, and the method includes running at least a portion of the keyboard simulator software module on the remote workstation, displaying a simulated keyboard on the review monitor, and using the keyboard simulator software module to communicate with the local workstation to thereby control the ultrasound system.

18. A method in accordance with claim 17 further comprising using a pair of keyboard/video/mouse (KVM) switches and a point-to-point local area network (LAN) to communicatively couple the local workstation to the remote workstation, and excluding control signals resulting from use of said visual keyboard simulator software module to control the ultrasound system from data communicated via said KVM switches and said LAN.

19. A method in accordance with claim 11 wherein the remote workstation comprises a remote physical keyboard, a pair of keyboard/video/mouse (KVM) switches, and a point-to-point cable, and the method further comprises using the pair of KVM switches and the point-to-point cable to communicate data from the remote physical keyboard to the remote workstation to thereby control the ultrasound system.

20. A physiology network in accordance with claim 11 wherein the ultrasonic system includes a local keyboard, and said remote physical keyboard includes a duplicate of all of the keys and controls provided by the local keyboard.
